(19) United States
(12) Patent Application Publication
(10) Pub. No.: US 2013/0281420 A1
(43) Pub. Date: Oct. 24, 2013

(54) Ophthalmic Formulations of Squalamine

(71) Applicants: Irach B. Taraporewala, White Plains, NY (US); Samuel I. Backenroth, New York, NY (US)

(72) Inventors: Irach B. Taraporewala, White Plains, NY (US); Samuel I. Backenroth, New York, NY (US)

(73) Assignee: Ohr Pharmaceutical, Inc., New York, NY (US)

(21) Appl. No.: 13/817,306

(22) PCT Filed: Aug. 16, 2011
(86) PCT No.: PCT/US11/47920

§ 371 Date: Jun. 24, 2013

Related U.S. Application Data
(60) Provisional application No. 61/374,524, filed on Aug. 17, 2010.

(51) Int. Cl. A61K 9/00; A61K 31/575

(52) U.S. Cl. CPC A61K 9/0048; A61K 31/575
USPC 514/182

(57) ABSTRACT

The invention relates to ophthalmic formulations of squalamine or its pharmaceutically acceptable salts for the treatment of conditions of the eye such as, for example, wet age-related macular degeneration (wet AMD), choroidal neovascularization, retinopathy, dry age-related macular degeneration (dry AMD), polypoidal choroidal vasculopathy, neovascularization following ocular surgery, macular edema, retinal venous occlusion, subchoroidal neovascularization, retinal epithelial detachment, pterygum or foveal geographic atrophy of the retinal pigment epithelium.

### CROSS REFERENCE TO RELATED APPLICATIONS

This application is technically related to U.S. Pat. No. 5,192,756 (issued Mar. 9, 1993), U.S. Pat. No. 6,962,909 (issued Nov. 8, 2005) and U.S. Pat. No. 7,981,876 (issued Jul. 19, 2011), each of which is incorporated by reference in its entirety.

### FIELD OF THE INVENTION

The invention relates to ophthalmic formulations of squalamine or its pharmaceutically acceptable salts for the treatment of conditions of the eye such as, for example, wet age-related macular degeneration (wet AMD), choroidal neovascularization, retinopathy, dry age-related macular degeneration (dry AMD), polypoidal choroidal vasculopathy, neovascularization following ocular surgery, macular edema, retinal venous occlusion, subchoroidal neovascularization, retinal epithelial detachment, pterygum or foveal geographic atrophy of the retinal pigment epithelium.

### BACKGROUND OF THE INVENTION

Age-related macular degeneration (AMD) is the leading cause of irreversible central vision loss among people in the United States aged 52 or older and is the most common overall cause of blindness in the United States, Canada, Great Britain and Australia. AMD encompasses several types of abnormalities that develop in the macula of affected individuals. Two forms of macular degeneration exist: dry (also known as atrophic) and wet (also known as disciform, exudative, subretinal neovascular or choroidal neovascular). The dry form, which may be a precursor to the wet form, results from an inability of the pigment epithelium of the macula to remove waste materials generated by the retina. The wet form occurs when new blood vessels grow under the retina, particularly the macula.

Squalamine (IUPAC Name: ([6-[(3S,5R,7R,10S,13R,145)-3-[3-(4-aminobutylamino)propylamino]-7-hydroxy-10,13-dimethyl-2,3,4,5,6,7,8,9,11,12,14,15,16,17-tetradecahydro-1H-cyclopenta[a]phenanthren-17-yl]-2-methylheptan-3-yl]hydrogen sulfate) is an aminosterol exhibiting anti-angiogenic properties that has been utilized as an intravenous infusion for the effective treatment of wet AMD where it functions to prevent the neovascularization and aberrant blood vessel formation in the retina that characterize the progression of the disease (Sills Jr. et al., “Squalamine Inhibits Angiogenesis and Solid Tumor Growth in Vivo Perturbs Embyronic Vasculature”, Jul. 1, 1998, Cancer Research, 58, 2784-2792; Higgins et al., “Squalamine Improves Retinal Neovascularization”, May 2000, Investigative Ophthalmology & Visual Science, vol. 41, No. 6, pp. 1507-1512.; PRNEWSWIRE, “Genaera Reports Squalamine Continues to Improve Vision at Four Months Timepoint in Age-Related Macular Degeneration”, Oct. 7, 2003, http://www.eyesightnews.com/topic/28.html). Squalamine is the subject of U.S. Pat. No. 5,192,756 to Zasloff et al., the disclosure of which is herein incorporated by reference in its entirety. The total chemical synthesis of squalamine is described in U.S. Pat. Nos. 6,262,283 and 6,610,866, which are incorporated herein by reference in their entirety.

It would clearly be desirable from a patient-use and risk standpoint to have available a topical formulation for direct application to the eye as opposed to an intravenous infusion, or especially the current standard of care which requires monthly injections directly into the eye. Topical formulations in the form of, for example, solutions, suspensions, creams or ointments are easily self-administered by patients as compared to more invasive techniques, such as intravenous infusions, which require costly administration under medical supervision and which can result in serious complications such as endophthalmitis and retinal detachment. The general problem with ocular eyedrops, however, is that after their administration, typically less than 5% of the drug in the eyedrop penetrates the cornea and reaches intraocular tissues. Instead, a major fraction of the administered dose is eliminated due to solution drainage and systemic absorption (Jarvinen K. et al., “Ocular absorption following topical delivery”, Adv. Drug Deliv. Rev. 1995; 16(1):3-19. See also Conroy C. W. , “Sulfonamides do not reach the retina in therapeutic amounts after topical application to the cornea”, Ocul. Pharmacol. Ther. 1997; 13(5):465-472 and Maurice D. M., “Drug delivery to the posterior segment from drops”, Surv. Ophthalmol. 2002; 47(suppl. 1):S41-S52).

In addition, a previous clinical trial to test the efficacy of squalamine for the treatment of AMD by IV infusion revealed potential problems for long term use. The intravenous dosing regimen in the IV formulation was deemed to be sub-optimal using pharmacokinetic analyses and was not viable on a commercial basis for a variety of reasons. For one, the short plasma half-life of squalamine in human subjects at the 40 mg dose resulted in concentrations in the choroid insufficient to block choroidal neovascularization (CNV) after 4-6 days. When the dosing was spaced out to monthly “maintenance” infusions, there was potentially only up to a week of inhibition of CNV, followed by three weeks or more of active new angiogenesis. This regimen produced good gains in visual acuity after the first four to five weeks of administration, followed by a decline in the rate of improvement after the fifth week. Intravenous dosing caused local infusion-site reactions (dosing was orders of magnitude higher than dose to be used in the topical formulation). In a “real world” situation, it is unrealistic to expect an elderly patient with wet AMD to be able to visit a clinic on a weekly basis for a prolonged infusion. Most retinal ophthalmic practices are also not set up for such intravenous infusions.

Compared to the above indicated disadvantages associated with intravenous dosing, the present invention represents the discovery of a safe and non-irritating ocular formulation for topical administration that is able to achieve selected delivery of a therapeutic agent to the back of the eye for treatment of a disorder.

### SUMMARY OF THE INVENTION

One aspect of the present invention is a composition for topical ophthalmic use comprising squalamine or a pharmaceutically acceptable salt thereof, one or more mucoadhesive agents and one or more penetration enhancers.

In another aspect of the invention, the composition further comprises at least one of a viscosity increasing agent, a tonicity modifier, an antimicrobial preservative, a buffering agent, a surfactant, a stabilizing agent, a solubilizing and a resuspension agent.

Another aspect of the present invention is a method for preventing and/or treating an ophthalmic condition comprising topically administering to the eye of a mammal, such as a human, in need thereof a therapeutically effective amount of squalamine or a pharmaceutically salt thereof.

In an exemplary embodiment, the ophthalmic condition is selected from the group consisting of wet age-related macular degeneration (wet AMD), choroidal neovascularization, retinopathy or dry age-related macular degeneration (dry AMD) and foveal geographic atrophy of the retinal pigment epithelium.

In an exemplary embodiment, the squalamine is present as the dilactate salt.

In an exemplary embodiment, the composition further comprises at least one of a non-ionic tonicity adjusting agent, a salt, a preservative, a buffering agent, a surfactant, a solubilizing agent and a stabilizer.

In an exemplary embodiment, the composition is administered topically.

In an exemplary embodiment, the composition is in the form of eye drops, a gel, lotion, cream, ointment, incorporated into a drug eluting ophthalmic conformer, an erodible ocular implant, a juxtascleral implant, a lacrimal stent, an iontophoresis ocular delivery system or an ophthalmic spray drug delivery device.

An aspect of the invention is a method of delivering squalamine or a pharmacologically acceptable salt thereof to the posterior sclera of the eye of a mammal in a therapeutically effective amount by administering a composition comprising squalamine or a pharmaceutically acceptable salt thereof; one or more mucoadhesive agents; and one or more penetration enhancers, while concomitantly producing negligible concentrations of the composition in the aqueous humor or vitreous humor.

In an exemplary embodiment, the mucoadhesive agent is selected from the group consisting of Carbopol 980, hydroxypropylmethylcellulose, Povidone K-30 and polyvinyl alcohol.

In an exemplary embodiment, the penetration enhancer is selected from the group consisting of n-dodecyl-β-D-maltoside, laurocapram and glycerol monolaurate, and PGML (polyethylene glycol monolaurate).

In an exemplary embodiment, the ophthalmic condition is wet AMD.

In an exemplary embodiment, the squalamine dilactate is present in an amount of 0.005 to 5.0 weight percent.

In an exemplary embodiment, the non-ionic tonicity adjusting agent is present in amount sufficient to generate a tonicity of about 50 to 350 milliosmols per kilogram.

In an exemplary embodiment, the salt is present in an amount sufficient to approximate the salt concentration and/or tonicity of the human tear fluid.

In an exemplary embodiment, the salt is present in an amount ranging from 0.3% to 1% weight percent.

In an exemplary embodiment, the preservative is present in an amount sufficient to generate a microbial barrier to maintain or reduce microbial concentrations for a period of from about 12 hours to about 72 hours.

### BRIEF DESCRIPTION OF THE FIGURES

The figures are only an illustrative embodiment of the scope of the present invention and are not intended to otherwise limit the scope of the invention.

FIG. 1 shows the disruption of Human Vascular Endothelial Cell (HUVEC) tube formation by squalamine.

### DETAILED DESCRIPTION OF THE INVENTION

In an exemplary embodiment, the ophthalmic formulations of the invention contain squalamine or a pharmaceutically acceptable salt thereof, a mucoadhesive agent and a penetration enhancer. The formulations may optionally also include, but are not limited to, at least one of (a) a tonicity modifier; (b) an antimicrobial preservative; (c) a buffering agent; (d) a surfactant; (e) a stabilizing agent; (f) a solubilizing or resuspension agent; (g) an additional mucoadhesive agent; and (h) an additional penetration enhancer.

The topical formulations of the invention are believed to target the back of the eye. For a topical formulation to be advantageous in targeting the back of the eye, it should have the properties of being able to reach the posterior sclera of the eye in sufficient concentrations. Ideally, the formulation should have enhanced residence time on the cornea without being flushed away by tears before diffusing to the rear of the eye, such as from the anterior sclera to the posterior sclera. Because a drug molecule may adversely affect the lens of the eye, such as by clouding it, the drug molecule should not pass from the front of the eye into the orb and enter the aqueous humor and vitreous humor within the eyeball to any significant degree. The formulations of the present invention possess the desired and unique characteristics needed to effectively deliver a drug molecule, such as squalamine or a pharmaceutically acceptable salt thereof, which is applied to the front of the eye to the rear of the eye where the therapeutic concentrations of the drug molecule are required for treatment of the targeted disorder. After administration onto the surface of the eye, the composition enter the conjunctiva and anterior sclera and into the corneal layer. The mucoadhesive agent is believed to increase residence time in the cornea so that the drug may diffuse slowly over time to the posterior sclera, resulting in delivery of sustained concentrations of squalamine or pharmaceutically acceptable salts thereof in the posterior sclera. The mucoadhesive agent accomplishes this objective by retarding the loss of the drug through, for example, drainage from the nasolachryimal duct due to lachrymation and tear turnover. The mucoadhesive agent also typically possesses viscosity enhancing properties that may result in a desirable soothing or lubricating effect. The penetration enhancer agent which is optionally added to the formulation enhances penetration of the formulation into the corneal epithelial layers, further enhancing the residence time of the squalamine or pharmaceutically acceptable salts thereof in the eye. The stabilizing agent may act as an antioxidant or otherwise retard the chemical degradation of the squalamine formulation. The buffering agent buffers the formulation to a comfortable near-neutral pH compatible with ocular administration. The tonicity modifier in the formulation produces the appropriate osmolality of the ophthalmic formulation.

The resulting formulations are stable, and after sterilization, may be packaged, stored and used directly. In an exemplary embodiment, the formulations are in drop form in the manner typically used to apply eye drops. The normal squeeze-type liquid drop application devices are perfectly suited for use in applying the ophthalmic formulations of the invention. In an exemplary embodiment, the formulations are conveniently administered by dropwise addition of the formulations into the affected eye(s) of the user.

The formulations of the present invention containing preservatives are especially advantageous for use in multi-dose containers. Multi-dose containers, as used herein, refer to containers which allow two or more separate applications of the ophthalmic formulation present within the container. Such containers are resealable—i.e., the container cap may be removed for a first application, and then the cap may be replaced onto the container, thereby providing a substantially liquid impermeable seal again. In an exemplary embodiment, an antimicrobial preservative is present in an amount sufficient to reduce microbial concentrations for a period of about 12 hours to about 72 hours, such as about 12 hours to about 48 hours, such as about 12 hours to about 24 hours.

In an exemplary embodiment, those formulations containing no preservative are packaged in a unit dose container—i.e., where only a single dose can be provided by a given container. Such preservative-free compositions are subject to uncontrolled microbial growth once the consumer initially breaks the container seal. Accordingly, the consumer is instructed to dispose of the container after the first dose. An appropriate unit-dose system such as blow-fill-seal unit dose preservative-free packaging system is typically used for the preservative-free formulations.

Pharmaceutical compositions for the topical ophthalmic administration of the squalamine or it salts thereof of this invention may be formulated in conventional ophthalmologically compatible vehicles, such as, for example, an ointment, cream, suspension, lotion, powder, solution, paste, gel, spray, aerosol or oil.

As used herein, the term “macular degeneration” is intended to encompass all forms of macular degeneration and includes a gradual loss of central vision usually affecting either or both eyes that occurs especially in the elderly. A slowly progressing form of macular degeneration, usually referred to as the dry form, is marked especially by the accumulation of yellow deposits in the macula lutea and the thinning of the macula lutea. A rapidly progressing form of macular degeneration, usually referred to as the wet form, is marked by scarring produced by bleeding and fluid leakage from new blood vessels formed below the macula lutea. Macular degeneration may exist as either the wet form or the dry form.

As defined herein, a “therapeutically effective amount” is an amount of an active agent (such as squalamine) which inhibits, totally or partially, the progression of the condition or alleviates, at least partially, one or more symptoms of the condition. A therapeutically effective amount can also be an amount that is prophylactically effective. The amount that is therapeutically effective will depend upon the patient's size and gender, the condition to be treated, the severity of the condition and the result sought. For a given patient, a therapeutically effective amount can be determined by methods known to those of skill in the art. The concentration of squalamine or a pharmaceutically acceptable salt thereof will typically be about 0.005 to about 5.0 weight percent, such as about 0.010 to about 4.0 weight percent, such as about 0.020 to about 3.0 weight percent, such as about 0.030 to about 2.0 weight percent, such as about 0.050 to about 1.0 weight percent.

In an exemplary embodiment, the squalamine is in the form of the dilactate salt. In an exemplary embodiment, the squalamine dilactate salt is employed at a concentration of about 0.1 to about 0.3% w/v, such as about 0.1 to 0.2% w/v.

Optionally, the formulations of the present invention contain a tonicity modifier.

In an exemplary embodiment, the tonicity modifier is non-ionic. The tonicity modifier may be selected from, but is not limited to, mannitol, sorbitol, dextrose, sucrose, urea, glycerol, polyethylene glycol and any mixtures thereof. In an exemplary embodiment, the tonicity modifier is present in amount sufficient to generate a tonicity of about 50 to about 350 milliosmols per kilogram (mOsmol/kg), such as about 65 to about 325 mOsmol/kg, such as about 80 to about 310 mOsmol/kg, such as about 95 to about 295 mOsmol/kg, such as about 110 to about 280 mOsmol/kg, such as about 125 to about 265 mOsmol/kg, such as about 140 to about 250 mOsmol/kg, such as about 155 to about 235 mOsmol/kg, such as about 170 to about 220 mOsmol/kg, such as about 185 to about 205 mOsmol/kg.

The formulation may also contain, an ionic salt, selected from, but not limited to, alkali metal halides (such as, for example, NaCl, KCl, NaBr, etc.), in an amount ranging from about 0.3% to about 1% weight percent or sufficient to approximate the salt concentration and/or tonicity of the human tear fluid. Selected salts from this group may also be referred to as ionic tonicity modifiers.

Where a preservative is used in the formulations of the present invention, an antimicrobial is present in an amount sufficient to generate a microbial barrier to maintain or reduce microbial concentrations for a period of about 12 hours to about 72 hours, such as about 12 hours to about 48 hours, such as about 12 hours to about 24 hours. Preservatives include, but are not limited to, benzalkonium chloride, benzyl alcohol, chlorobutanol, cetrimonium, methylparaben, propylparaben, polyamino propyl biguanide, phenylethyl alcohol, chlorohexidine, chlorohexidine digluconate, chloroquat, stabilized oxychloro complex or any combination thereof.

Buffering agents that can be used in the formulations of the present invention include, but are not limited to, buffers prepared from sodium, potassium bicarbonate, phosphate, acetate, citrate, borate salts and/or phosphoric acid, acetic acid, citric acid or boric acid. In an exemplary embodiment, the buffer is sodium dihydrogen phosphate or disodium phosphate or boric acid/sodium borate. The buffers of the invention should be present in an amount sufficient to produce and maintain a product pH of about 5.5 to about 8.0, such as about 5.7 to about 7.7, such as about 6.0 to about 7.4, such as about 6.3 to about 7.1, such as about 6.6 to about 6.8, and including a pH of about 5.7, about 5.9, about 6.1, about 6.3, about 6.5, about 6.7, about 6.9, about 7.1, about 7.3, about 7.5, about 7.7 or about 7.9.

A surfactant may also be added to the formulations of the present invention. In an exemplary embodiment, the surfactant is present at a concentration range of about 0.001% to about 0.3%, such as about 0.005% to about 0.2%, such as about 0.01% to about 0.1%, such as about 0.05% to about 0.1%, to provide enhanced wetting characteristics to the formulation. The surfactant may include, but is not limited to, poloxamers, polysorbate 80, polysorbate 20, tyloxapol, polyoxoethylene, Brij 35, Brij 58, Brij 78, Aptet 100, G 1045, Spans 20, 40 and 85, Tweens 20, 40, 80 or 81, sodium lauroyl sarcosinate, lauroyl-L-glutamic acid triethanolamine, sodium myristyl sarcosinate and sodium lauryl sulfate., polyoxyethylenesorbitan fatty acid esters, polyoxyethylenehydrogenated castor oil, polyethyleneglycol fatty acid esters (e.g., polyoxyl stearate), polyoxyethylenepolyoxypropylene alkyl ethers, polyoxyalkylene alkyl phenyl ethers, polyglycerol fatty acids esters (e.g., decaglyceryl monolaureate), glycerol fatty acid esters, sorbitan fatty acid esters, and polyoxyethylenepolyoxypropylene glycol (poloxamer), decaglyceryl monolaureate, polyoxyl stearate 40, and polyoxyethylenehydrogenated castor oil, or any combination thereof.

A stabilizer can also be added to the formulations of the present invention. Suitable stabilizers include, but are not limited to, sodium metabisulfite, sodium bisulfate, acetylcysteine, ascorbic acid, sodium thiosulfate, alpha-tocopherol, carnosine, retinyl palmitate, salts of ethylenediaminetetraacetic acid (EDTA) (such as, for example, the disodium, tetrasodium, calcium or calcium sodium edetate salts), or any combination thereof.

The mucoadhesive agent present in the described formulations increases corneal contact time, enhances bioavailability and/or produces a lubricating effect, and includes, but is not limited to, acrylic acid polymers, methylcellulose, ethylcellulose, hydroxypropylmethyl cellulose, hydroxyethylcellulose, Carbopol® polymers (such as, for example, Carbopol® 674, 676, 690, 980 NF, ETD-2691, ETD 2623, EZ-2, EZ-3, EZ-4, Aqua 30 and Novethix™ L-10), hydroxypropylcellulose, polyvinyl alcohol, cellulose acetate phthalate, alginate, gelatin, sodium chondroitin sulfate, or any combination thereof.

The penetration enhancer present in the described formulations includes, but is not limited to, laurocapram (azone), bile acids and their alkali metal salts, including chenodeoxycholoc acid, cholic acid, taurocholic acid, taurodeoxycholic acid, tauroursodeoxycholic acid or ursodeoxycholic acid, glycocholate, n-dodecyl-β-D-maltoside, sucrose dodecanoate, octyl maltoside, decyl maltoside, tridecyl maltoside, tetradecyl maltoside, hexamethylene lauramide, hexamethylene octanamide, glycerol monolaurate, PGML (polyethylene glycol monolaurate), dimethyl sulfoxide, methylsulfonylmethane, sodium fusidate, saponins or any combination thereof.

In addition, a solubilizing or resuspension agent may also be added to the formulations of the present invention. Suitable solubilizing or resuspension agents include, but are not limited to, cyclodextrins (CDs), such as hydroxypropyl gamma-CD (Cavasol®), sulfobutyl ether 4 beta-CD (Captisol®), and hydroxypropyl beta-CD (Kleptose®), Polysorbate 80 (Tween80®) or hyaluronic acid or hyaluronate salts. The cyclodextrins in particular may also exhibit permeation enhancing properties.

Pharmaceutically acceptable salts of squalamine include, but are not limited to, acid addition salts such as acetate, adipate, benzoate, benzenesulfonate, citrate, camphorate, decanoate, dodecylsulfate, heptanoate, hydrochloride, hydrobromide, lactate, maleate, methanesulfonate, nitrate, oleate, oxalate, palmitate, phosphate, pivalate, propionate, succinate, sulfate, tartrate, toluene-p-sulfonate; and undecanoate; and base salts such as ammonium salts, alkali metal salts such as sodium and potassium salts, alkaline earth metal salts such as calcium and magnesium salts, salts with organic bases such as dicyclohexylamine salts and salts with amino acids such as arginine.

Both mono- and di-salts of squalamines are intended to be included as suitable salts for the formulations of the invention. As an example, both the monolactate and dilactate salts of squalamine would be included.

In a particular embodiment, the salt is the dilactate salt. The dilactate salt of squalamine exists in an amorphous form or in a crystalline form. In an exemplary embodiment of the invention, the crystalline form of the dilactate salt exists as a solvate. In another exemplary embodiment, the crystalline form exists as a hydrate, and in a further embodiment the dilactate salt exists as a solvate and a hydrate. The crystalline forms of squalamine dilactate may exist as solvates, where solvent molecules are incorporated within the crystal structure. As an example, when the solvent contains ethanol, the crystal may contain ethanol molecules. In another embodiment, the solvate may contain water, and the crystal may be a hydrate containing water in the crystal structure. In another embodiment, the crystal may be both a solvate and a hydrate. A discussion of the various crystalline forms of squalamine dilactate may be found in U.S. Pat. No. 7,981,876, which is incorporated by reference in its entirety.

For an exemplary listing of typical carriers, stabilizers and adjuvants known to those of skill in the art that may be useful in the ophthalmic compositions described herein, see Gennaro (2005) Remington: The Science and Practice of Pharmacy, Mack Publishing, 21^{st} ed.

In vivo administration of the squalamine-containing compositions of the invention may be effected in one dose, multiple doses, continuously or intermittently throughout the course of treatment. Methods of determining the most effective dosage of administration are well known to those of skill in the art and will vary with the composition used for therapy, the purpose of the therapy and the subject being treated. Single or multiple administrations can be carried out with the dose level and pattern being selected by the treating physician.

In a particular embodiment, the pH of the solution is in the range of about 7.0 to about 7.5. In an exemplary embodiment, the solution is preferably a hypotonic solution. In a particular embodiment, the pH is about 7.2 to about 7.4.

In various exemplary embodiments, the topical formulations of the present invention include, but are not limited to, ointments, gels, creams or eye drops.

Various particularized and non-limiting formulations are listed as follows:

squalamine dilactate+n-dodecyl-β-D-maltoside+Povidone K-30+phosphate buffer;

squalamine dilactate+n-dodecyl-β-D-maltoside+3-hydroxypropyl-β-cyclodextrin+Povidone K-30 +phosphate buffer;

squalamine dilactate+n-dodecyl-β-D-maltoside+Carbopol 980+borate buffer;

squalamine dilactate+n-dodecyl-β-D-maltoside+Carbopol 980+phosphate buffer;

Various particularized and non-limiting formulations described in the examples below. These formulations are merely illustrative of the described invention and are not intended to limit the scope of the described invention.

### EXAMPLES

### Example 1

### Formulation A

This formulation contained 0.2% squalamine dilactate as active drug, 67 mM NaH₂PO₄+Na₂HPO₄ (0.9%) as buffer, NaCl (˜0.4%) as tonicity modifier, edetate disodium (0.01%) as chelating agent /stabilizer, benzalkonium chloride (0.005%) as preservative and a sufficient quantity of water for injection or purified water USP.

Formulation A was prepared as follows: 50 mL of purified water was placed in a 250 mL graduated glass beaker with a stir bar; 2.688 g of sodium phosphate heptahydrate was added to the beaker and stirred until it dissolved; 1.24 g of sodium phosphate monobasic monohydrate was added to the beaker and stirred until it dissolved; 0.400 g of sodium chloride was added to the beaker and stirred until it dissolved; 0.005 g of benzalkonium chloride was added to the beaker and stirred until it dissolved; 0.01 g of disodium EDTA was added to the beaker and stirred until it dissolved; 0.200 g of squalamine dilactate was added to the beaker and stirred until it dissolved; about 40 mL of purified sterile water was added to the beaker; the pH was adjusted to 7.2 using 2 N NaOH and 1 N HCl (when necessary); the volume was sufficient quantity of water for injection or purified water USP.

### Example 2

### Formulation B

This formulation contained 0.2% squalamine dilactate as active drug, 67 mM NaH₂PO₄+Na₂HPO₄ (0.9%) as buffer, NaCl (˜0.4%) as tonicity modifier, edetate disodium (0.01%) as chelating agent /stabilizer, Carbopol 980 NF (0.5%) as a mucoadhesive agent and a sufficient quantity of water for injection or purified water USP.

Formulation B was prepared as follows: 50 mL of purified water was placed in a 250 mL graduated glass beaker with a stir bar; 2.688 g of sodium phosphate heptahydrate was added to the beaker and stirred until it dissolved; 1.24 g of sodium phosphate monobasic monohydrate was added to the beaker and stirred until it dissolved; 0.400 g of sodium chloride was added to the beaker and stirred until it dissolved; 0.01 g of disodium EDTA was added to the beaker and stirred until it dissolved; 0.200 g of squalamine dilactate was added to the beaker and stirred until it dissolved; 0.500 g of Carbopol 980 NF was added to the beaker and stirred until it dissolved; about 40 mL of purified sterile water was added to the beaker; the pH was adjusted to 7.2 using 2 N NaOH and 1 N HCl (when necessary); the volume was made up to 100 mL; and the solution was filtered using a sterile filtration assembly using a 0.22 micron filter.

### Example 3

### Formulation C

This formulation contained 0.2% squalamine dilactate as active drug, 67 mM NaH₂PO₄+Na₂HPO₄ (0.9%) as buffer, mannitol (˜0.8%) as tonicity modifier, edetate disodium (0.01%) as chelating agent /stabilizer, Carbopol 980 NF (0.5%) as a mucoadhesive agent, n-dodecyl-β-D-maltoside (0.05-0.1%) as a penetration enhancer, benzalkonium chloride (0.005%) as preservative and a sufficient quantity of water for injection or purified water USP.

Formulation C was prepared as follows: 50 mL of purified water was placed in a 250 mL graduated glass beaker with a stir bar; 2.688 g of sodium phosphate heptahydrate was added to the beaker and stirred until it dissolved; 1.24 g of sodium phosphate monobasic monohydrate was added to the beaker and stirred until it dissolved; 0.800 g of mannitol was added to the beaker and stirred until it dissolved; 0.005 g of benzalkonium chloride was added to the beaker and stirred until it dissolved; 0.01 g of disodium EDTA was added to the beaker and stirred until it dissolved; 0.500 g of Carbopol 980 NF was added to the beaker and stirred until it dissolved; 0.200 g of squalamine dilactate was added to the beaker and stirred until it dissolved; 0.05 g of n-dodecyl-β-D-maltoside was added to the beaker and stirred until it dissolved; about 40 mL of purified sterile water was added to the beaker; the pH was adjusted to 7.2 using 2 N NaOH and 1 N HCl (when necessary); the volume was made up to 100 mL; and the solution was filtered using a sterile filtration assembly using a 0.22 micron filter.

### Example 4

### Formulation D

This formulation contained 0.1% squalamine dilactate as active drug, 50 mM NaH₂PO₄+Na₂HPO₄ (0.9%) as buffer, NaCl (˜0.9%) as tonicity modifier, edetate disodium (0.01%) as chelating agent /stabilizer, hydroxypropyl-methylcellulose as a mucoadhesive agent, chenodeoxycholic acid (0.005%) as an penetration enhancer, benzalkonium chloride (0.005%) as preservative and a sufficient quantity of water for injection or purified water USP. This formulation was prepared in a manner similar to the formulations above.

### Example 5

### Formulation E

This formulation contained 0.2% squalamine dilactate as active drug, 67 mM NaH₂PO₄+Na₂HPO₄ (0.9%) as buffer, NaCl (˜0.4%) as tonicity modifier, edetate disodium (0.01%) as chelating agent /stabilizer, hydroxypropyl-methylcellulose as a mucoadhesive agent and a sufficient quantity of water for injection or purified water USP.

This formulation was prepared in a manner similar to the formulations above.

### Example 6

### Formulation F

This formulation contained 0.1% squalamine dilactate as active drug, boric acid (0.8%)+sodium borate (0.12%) as buffer, mannitol (˜0.8%) as tonicity modifier, alpha-tocopherol (0.005%) as chelating agent/stabilizer, Carbopol 980 NF (0.5%) as a mucoadhesive agent, n-dodecyl-β-D-maltoside (0.05-0.1%) as a penetration enhancer, benzalkonium chloride (0.005%) as a preservative and a sufficient quantity of water for injection or purified water USP.

This formulation was prepared in a manner similar to the formulations above.

### Example 7

### Formulation G

This formulation contained 0.2% squalamine dilactate as active drug, sodium phosphate heptahydrate 1.88% w/v and sodium phosphate monobasic monohydrate 1.0% w/v as buffers, povidone K-30 1.2% w/v as emollient, edetate disodium 0.01% as stabilizing agent, n-dodecyl-β-D-maltoside 0.005% w/v as permeation enhancer, benzalkonium Chloride 0.005% w/v as preservative, 3-hydroxypropyl-B-cyclodextrin 0.9% w/v as solubilizing agent and purified water qs. The pH=6.70 and the osmolality=315 mOsm/kg. The solution was sterile filtered through 0.22 micron filter before use.

This formulation was prepared in a manner similar to the formulations above.

### Example 8

### Formulation H

This formulation contained 0.2% squalamine dilactate as active drug, glycerin 1% w/v as emollient, boric Acid 1.18% w/v and sodium borate 0.12% w/v as buffers, n-dodecyl-β-D-maltoside 0.005% w/v as permeation enhancer, benzalkonium chloride 0.005% as preservative and purified water qs. The pH=6.90 and the osmolality=305 mOsm/kg. The solution was sterile filtered through 0.22 micron filter before use.

This formulation was prepared in a manner similar to the formulations above.

### Example 9

### Formulation I

This formulation contained 0.2% squalamine dilactate as active drug, sodium phosphate heptahydrate 1.88% w/v and sodium phosphate monobasic monohydrate 0.87% w/v as buffers, sodium chloride 0.3% w/v as tonicity modifier, edetate disodium 0.01% stabilizing agent, benzalkonium chloride 0.005% w/v as preservative, 3-hydroxypropyl-B-cyclodextrin 0.9% w/v as solubilizing agent and purified water qs. The pH=6.72 and the osmolality=325 mOsm/kg. The solution was sterile filtered through 0.22 micron filter before use.

This formulation was prepared in a manner similar to the formulations above.

### Example 10

### Formulation J

This formulation contained 0.2% squalamine dilactate as active drug, sodium phosphate heptahydrate 1.88% w/v and sodium phosphate monobasic monohydrate 1.0% w/v as buffers, providone K-30 0.6% w/v as emollient, edetate disodium 0.01% as stabilizing agent, n-dodecyl-β-D-maltoside 0.005% w/v as permeation enhancer, benzalkonium chloride 0.005% w/v as preservative and purified water qs. The pH=6.70 and the osmolality=295 mOsm/kg. The solution was sterile filtered through 0.22 micron filter before use.

This formulation was prepared in a manner similar to the formulations above.

### Example 11

### Formulation K

This formulation contained 0.2% squalamine dilactate as active drug, glycerin 1.0% w/v as emollient, mannitol 0.05% w/v as tonicity modifier, boric acid 1.18% w/v and sodium borate 0.12% w/v as buffers, sodium chloride 0.4% w/v as tonicity modifier, n-dodecyl-β-D-maltoside 0.005% w/v as permeation enhancer, benzalkonium chloride 0.005% w/v as preservative and purified water qs. The pH=5.86 and the osmolality=285 mOsm/kg. The solution was sterile filtered through 0.22 micron filter before use.

This formulation was prepared in a manner similar to the formulations above.

### Example 12

Stability Study on Squalamine Lactate Formulations

Formulations G and H (see above) were tested for stability for 2 weeks, 1 month, 3 months and 6 months at room temperature and at 40° C. The squalamine lactate concentration was assessed by HPLC at each time point (Table 1) and the stability of the formulation was assessed by visual observation and pH (Table 2). Squalamine lactate and the formulations were found to be stable at all time points.

TABLE 1
HPLC Analysis of Squalamine Lactate Content

| | | | | | | Squalamine | |
|---|---|---|---|---|---|---|---|
| | | Squalamine | | Squalamine | | amount | Squalamine |
| | Initial | amount | Squalamine | amount | Squalamine | at 3 | amount |
| | amount | at 2 weeks, | amount | at 1 month, | amount | months, | at 3 |
| | of | room | at 2 weeks, | room | at 1 month, | room | months, |
| | squalamine | temperature | 40° C. | temperature | 40° C. | temperature | 40° C. |
| Formulation | (mg/mL) | (mg/mL) | (mg/mL) | (mg/mL) | (mg/mL) | (mg/mL) | (mg/mL) |
| G | 1.92 | 1.93 | 1.93 | 1.96 | 1.92 | 1.98 | 1.90 |
| H | 1.95 | 1.96 | 1.94 | 1.95 | 1.95 | 1.97 | 1.93 |

| | Squalamine | |
|---|---|---|
| | amount | Squalamine |
| | at 6 months, | amount |
| | room | at 6 months, |
| | temperature | 40° C. |
| Formulation | (mg/mL) | (mg/mL) |
| G | 1.96 | 1.98 |
| H | 1.98 | 1.98 |

TABLE 2
Stability of Squalamine Lactate Formulations

| | | 2 weeks, | | 1 month, | | 3 months, | |
|---|---|---|---|---|---|---|---|
| | | room | 2 weeks, | room | 1 month, | room | 3 months, |
| Formulation | Initial | temperature | 40° C. | temperature | 40° C. | temperature | 40° C. |
| G | | | | | | | |
| Appearance | Clear | Clear | Clear | Clear | Clear | Clear | Clear |
| | colorless | colorless | colorless | colorless | colorless | colorless | colorless |
| | solution | solution free of | solution | solution free | solution | solution free | solution free |
| | free of | particulates | free of | of | free of | of | of |
| | particulates | | particulates | particulates | particulates | particulates | particulates |
| pH | 6.72 | 6.74 | 6.75 | 6.71 | 6.74 | 6.73 | 6.75 |
| H | | | | | | | |
| Appearance | Clear | Clear | Clear | Clear | Clear | Clear | Clear |
| | colorless | colorless | colorless | colorless | colorless | colorless | colorless |
| | solution | solution free | solution | solution free | solution | solution free | solution free |
| | free of | of | free of | of | free of | of | of |
| | particulates | particulates | particulates | particulates | particulates | particulates | particulates |
| pH | 6.90 | 6.88 | 6.91 | 6.86 | 6.90 | 6.91 | 6.92 |

| | 6 months, room | 6 months, |
|---|---|---|
| Formulation | temperature | 40° C. |
| G | | |
| Appearance | Clear colorless | Clear colorless |
| | solution free of | solution free of |
| | particulates | particulates |
| pH | 6.72 | 6.74 |
| H | | |
| Appearance | Clear colorless | Clear colorless |
| | solution free of | solution free of |
| | particulates | particulates |
| pH | 6.88 | 6.93 |

### Example 13

Tolerance Study of Squalamine Lactate Formulations by Topical Administration to the Rabbit Eye

Squalamine Lactate Formulation G and Squalamine Lactate Formulation H (see formulations above) were evaluated for ocular tolerance when given as a single daily dose by topical ocular instillation for 28 consecutive days to Dutch-belted rabbits. The vehicle controls were the squalamine lactate formulations without the squalamine lactate.

The study design was as follows:

Experimental Design

| | Dose Level | Concentration | Dose Volume | No. of |
|---|---|---|---|---|
| Identification | (ug/kg)^{a} | (mg/mL) | (μL/eye)^{b} | Animals |
| Vehicle | 0 | 0 | 50 | 3 |
| Control A | | | | |
| Squalamine | 38.4 | 1.92 | 20 | 3 |
| Lactate | | | | |
| Formulation G | | | | |
| Squalamine | 57.6 | 1.92 | 30 | 3 |
| Lactate | | | | |
| Formulation G | | | | |
| Squalamine | 96 | 1.92 | 50 | 3 |
| Lactate | | | | |
| Formulation G | | | | |
| Vehicle | 0 | 0 | 50 | 3 |
| Control B | | | | |
| Squalamine | 39 | 1.95 | 20 | 3 |
| Lactate | | | | |
| Formulation H | | | | |
| Squalamine | 58.5 | 1.95 | 30 | 3 |
| Lactate | | | | |
| Formulation H | | | | |
| Squalamine | 97.5 | 1.95 | 50 | 3 |
| Lactate | | | | |
| Formulation H | | | | |

^{a}Based on a 2 kg rabbit.
^{b}Doses given as once daily topical ocular instillation to each eye.

The following parameters and end points were evaluated in this study: clinical signs, body weights, body weight changes, ophthalmology, intraocular pressure, gross ocular examinations, gross necropsy findings and histopathologic examinations. There were no observed deaths and no treatment-related effects on body weights and body weight gains. There were also no treatment-related ophthalmologic findings, no effects on intraocular pressure and no macroscopic or microscopic findings. Based on these observations, formulations were safe and exhibited no signs of ocular toxicity.

Treatment-related eye redness and/or discharge and rarely swelling were noted in the eye(s) of animals given ≧38.4 μg/kg/day Squalamine Lactate Formulation G and/or ≧39 μg/kg/day Squalamine Lactate Formulation H and/or Vehicle Control B with a general increased incidence in animals given Squalamine Lactate Formulation H. Observations of discharge correlated with minor clinical signs of clear discharge noted on Day 14 in animals given both Squalamine Lactate formulations and animals given Vehicle Control B. These observations were considered non-adverse given their low severity (generally, very slight or any deviation from normal) and lack of ophthalmological, macroscopic or microscopic correlates.

In conclusion, administration of Squalamine Lactate Formulation G at 0, 38.4, 57.6 and 96 pg/kg/day and Squalamine Lactate Formulation H at 0, 39, 58.5 and 97.5 μg/kg/day by once daily topical ocular instillation was generally well-tolerated in Dutch-belted rabbits. Based on these results, the no-observed-adverse-effect level (NOAEL) was considered to be 96 μg/kg/day (Squalamine Lactate Formulation G) or 97.5 μg/kg/day (Squalamine Lactate Formulation H), and based on the incidence of minor ocular findings of redness and discharge at all doses of Squalamine Lactate Formulation H and occasionally in animals given Vehicle Control B, the Squalamine Lactate Formulation G and Vehicle Control A were considered better tolerated than Squalamine Lactate Formulation H and Vehicle Control B.

### Example 14

Ocular Biodistribution Study of a Squalamine Lactate Formulation in the Dutch-Belted Rabbit Following Ocular Administration

The objective of this study was to determine the ocular biodistribution of squalamine lactate formulation G (see composition above) when given once by ocular administration to male Dutch-Belted rabbits.

The study design was as follows:

Experimental Design

| Time | Route of | Dose | Concentration | Dose | No. of |
|---|---|---|---|---|---|
| Point | Administration | Level | (mg/mL) | Volume | Animals |
| 15 min. | Ocular | 0.08 | 1.92 | 40 | 3 |
| | instillation | mg/eye | | uL/eye^{a} | |
| 30 min | Ocular | 0.08 | 1.92 | 40 | 3 |
| | instillation | mg/eye | | uL/eye^{a} | |
| 3 hrs | Ocular | 0.08 | 1.92 | 40 | 3 |
| | instillation | mg/eye | | uL/eye^{a} | |

^{a}Dose was given once as a topical ocular instillation to each eye.

Body weight measurements were taken for randomization/dose calculation purposes. No treatment-related clinical signs were observed following ocular administration. Following dose administration, blood samples were collected at specified time points and plasma was prepared. After blood sample collection, the animals were euthanized and a necropsy was performed to collect the following ocular tissues: aqueous humor, vitreous humor, sensory retina and choroid/sclera. The plasma and ocular tissues were analyzed and the results of these analyses are indicated in the table below.

Squalamine in Rabbit Tissue Results (ng/gm)
Posterior Sclera and Choroid
Group 1 Sclera (0.08 mg/eye) 15 min

| | | |
|---|---|---|
| An#101 | An#102 | An#103 |

| left | right | left | right | left | right | Mean |
|---|---|---|---|---|---|---|
| 101 | 26.6 | 44.2 | 145 | 195 | 31.7 | 90.6 |

Group 1 Sclera (0.08 mg/eye) 30 min

| | | |
|---|---|---|
| An#104 | An#105 | An#106 |

| left | right | left | right | left | right | |
|---|---|---|---|---|---|---|
| 54.4 | 121 | BQL | 78.9 | 96.7 | 94.1 | 89.0 |

Group 1 Sclera (0.08 mg/eye) 3 hrs

| | | |
|---|---|---|
| An#107 | An#108 | An#109 |

| left | right | left | right | left | right | |
|---|---|---|---|---|---|---|
| 23.9 | 149 | 26.0 | 34.6 | 71.8 | 51.1 | 59.4 |

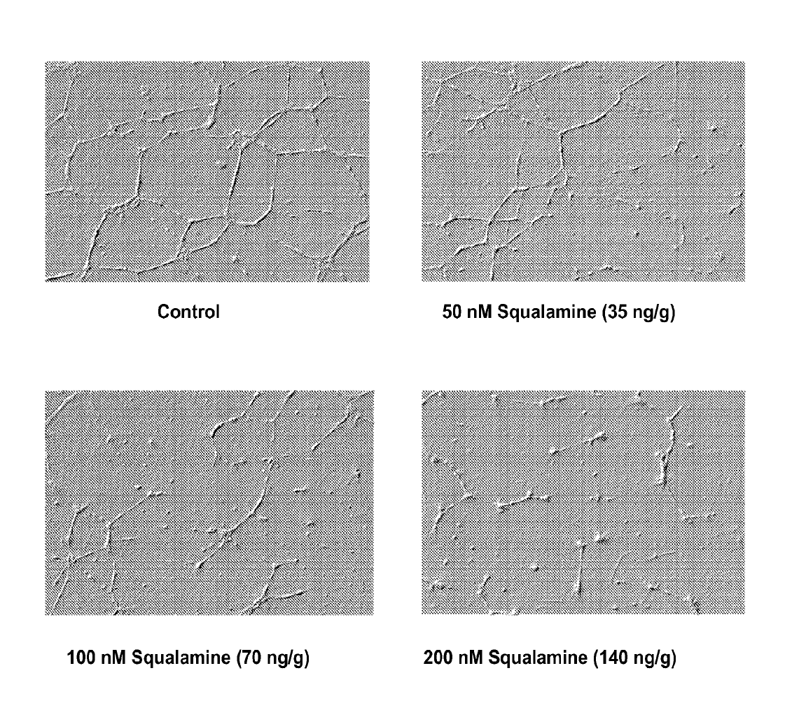

No quantifiable levels of squalamine were detected in the aqueous or vitreous humor of any animals, confirming that squalamine does not significantly penetrate through all the layers of the cornea or contact the lens. In conclusion, the results of the analysis of ocular tissues for the amount of squalamine lactate present show levels in the posterior sclera and choroid sufficient to disrupt HUVAC tube formation even at the three-hour time point (see FIG. 1 and Example 15 below). It can therefore be inferred (see, e.g., Invest. Ophthalmol. Vis. Sci. February 2005 vol. 46 no. 2 454-460 and US patent application publication #2010/0272719) that these levels are sufficient to block the deleterious choroidal neovascularization (CNV) process that occurs in wet-AMD.

### Example 15

Inhibition of VEGF Induced Tube Formation by HUVEC with Squalamine

Squalamine lactate was mixed in solution at 50, 100 or 200 nM concentration with a suspension of human vascular endothelial cells (HUVEC). The suspension was then immediately plated on Matrigel which contained multiple growth factors including vascular endothelial cell growth factor (VEGF). The plates were incubated at 37° C. in an atmosphere of 95% O₂/5% CO₂ for 24 hrs and then the plates were photographed. The results are shown in FIG. 1 and indicate that squalamine disrupts tube formation even at 50 nM concentration.

A number of references have been cited, the entire disclosures of which are incorporated herein by reference in their entirety.

1. A method of preventing or treating an ophthalmic condition in a mammal in need thereof, comprising administering to the eye of the mammal a therapeutically effective amount of a composition comprising
squalamine or a pharmaceutically acceptable salt thereof;
one or more mucoadhesive agents; and
one or more penetration enhancers,
wherein the ophthalmic condition is selected from the group consisting of wet age-related macular degeneration (wet AMD), choroidal neovascularization, retinopathy or dry age-related macular degeneration (dry AMD) and foveal geographic atrophy of the retinal pigment epithelium.

2. The method according to claim 1, wherein the squalamine is present as the dilactate salt.

3. The method according to claim 1, wherein the composition is administered topically.

4. The method according to claim 1, wherein the composition is in the form of eye drops, a gel, lotion, cream, ointment, incorporated into a drug eluting ophthalmic conformer, an erodible ocular implant, a juxtascleral implant, a lacrimal stent, an iontophoresis ocular delivery system or an ophthalmic spray drug delivery device.

5. The method according to claim 1, wherein the mammal is a human.

6. A method of delivering squalamine or a pharmacologically acceptable salt thereof to the posterior sclera of the eye of a mammal in a therapeutically effective amount by administering a composition comprising
squalamine or a pharmaceutically acceptable salt thereof;
one or more mucoadhesive agents; and
one or more penetration enhancers,
while concomitantly producing negligible concentrations of the composition in the aqueous humor or vitreous humor.

7. The method according to claim 6, wherein the composition is administered topically.

8. The method according to claim 1, wherein the mucoadhesive agent is selected from the group consisting of Carbopol 980, hydroxypropylmethylcellulose, Povidone K-30 and polyvinyl alcohol.

9. The method according to claim 1, wherein the penetration enhancer is selected from the group consisting of n-dodecyl-β-D-maltoside, laurocapram and glycerol monolaurate, and PGML (polyethylene glycol monolaurate).

10. The method according to claim 1, wherein the ophthalmic condition is wet AMD.

11. The method according to claim 1, wherein the composition further comprises at least one of a non-ionic tonicity adjusting agent, a salt, a preservative, a buffering agent, a surfactant, a solubilizing agent and a stabilizer.

12. An ophthalmic composition, comprising:
squalamine or a pharmaceutically acceptable salt thereof;
one or more mucoadhesive agents; and
one or more penetration enhancers.

13. The composition according to claim 12, further comprising at least one of a non-ionic tonicity adjusting agent, a salt, a preservative, a buffering agent, a surfactant, a solubilizing agent and a stabilizer.

14. The composition according to claim 12, where the squalamine is present as the dilactate salt.

15. The composition according to claim 12, wherein the composition is administered topically to the eye.

16. The composition according to claim 14, wherein the squalamine dilactate is present in an amount of 0.005 to 5.0 weight percent.

17. The composition according to claim 13, wherein the non-ionic tonicity adjusting agent is present in amount sufficient to generate a tonicity of about 50 to 350 milliosmols per kilogram.

18. The composition according to claim 13, wherein the salt is present in an amount sufficient to approximate the salt concentration and/or tonicity of the human tear fluid.

19. The composition according to claim 13, wherein the salt is present in an amount ranging from 0.3% to 1% weight percent.

20. The composition according to claim 13, wherein the preservative is present in an amount sufficient to generate a microbial barrier to maintain or reduce microbial concentrations for a period of from about 12 hours to about 72 hours.
